SEQUENCE LISTING (9 sequences)

SEQ ID NO: 1 - protein, 398 aa, Homo sapiens
MEGAALLRVSVLCIWMSALFLGVRVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 2 - protein, 396 aa, Homo sapiens
  MISC_FEATURE (1)..(396): Apo L 1 variant
MEGAALLRVSVLCIWMSALFLGVRVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNKILQADQEL

SEQ ID NO: 3 - protein, 398 aa, Homo sapiens
  MISC_FEATURE (1)..(398): Apo L 1 Variant
MEGAALLRVSVLCIWMSALFLGVRVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVGFFLVLDVVYLVYESKHLHEGAKSETAEELKKVAQELEEKLNMLNNNYKILQADQEL

SEQ ID NO: 4 - protein, 398 aa, Homo sapiens
  MISC_FEATURE: NP 003652.2
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 5 - protein, 396 aa, Homo sapiens
  MISC_FEATURE (1)..(396): Apo L 1 Variant
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNKILQADQEL

SEQ ID NO: 6 - protein, 398 aa, Homo sapiens
  MISC_FEATURE (1)..(398): Apo L 1 Variant
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVGFFLVLDVVYLVYESKHLHEGAKSETAEELKKVAQELEEKLNMLNNNYKILQADQEL

SEQ ID NO: 7 - protein, 398 aa, Homo sapiens
MEGAALLRVSVLCIWMSALFLGVRVRAEEAGARVQQNVPSGTDTGDPQSKPLGDWAAGTMDPESSIFIEDAIKYFKEKVSIQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 8 - protein, 396 aa, Homo sapiens
  MISC_FEATURE (1)..(396): Apo L 1 Variant
MEGAALLRVSVLCIWMSALFLGVRVRAEEAGARVQQNVPSGTDTGDPQSKPLGDWAAGTMDPESSIFIEDAIKYFKEKVSIQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNKILQADQEL

SEQ ID NO: 9 - protein, 398 aa, Homo sapiens
  MISC_FEATURE (1)..(398): Apo L 1 variant
MEGAALLRVSVLCIWMSALFLGVRVRAEEAGARVQQNVPSGTDTGDPQSKPLGDWAAGTMDPESSIFIEDAIKYFKEKVSIQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVGFFLVLDVVYLVYESKHLHEGAKSETAEELKKVAQELEEKLNMLNNNYKILQADQEL

(19) United States
(12) Patent Application Publication
(10) Pub. No.: US 2012/0128682 A1
(43) Pub. Date: May 24, 2012

(54) APOLIPOPROTEIN L- I VARIANTS AND THEIR USE

(75) Inventors: Etienne Pays, Nil-Saint-Vincent (BE); Laurence Lecordier, Fontaine L'Eveque (BE); Benolt Vanhollebeke, La Hulpe (BE)

(73) Assignee: Universite Libre de Bruxelles, Bruxelles (BE)

(21) Appl. No.: 13/388,645

(22) PCT Filed: Aug. 18, 2010
(86) PCT No.: PCT/EP2010/062065

§ 371 Date: Feb. 2, 2012

Related U.S. Application Data
(60) Provisional application No. 61/323,734, filed on Apr. 13, 2010.
(60) Provisional application No. 61/323,727, filed on Apr. 13, 2010.

(30) Foreign Application Priority Data
Aug. 18, 2009 (EP) PCT EP2009 060687

(51) Int. Cl. A61K 39/395; C07H 21/04; C12N 15/63; A61K 38/00; A61K 31/7088; A61P 3/06; A61P 33/00; C12Q 1/04; G01N 33/53; C12Q 1/68; C07K 16/12; C12N 5/10; C07K 16/18; A01K 67/027; A61K 35/14; A61K 35/16; C07K 14/775

(52) U.S. Cl. 424/139.1; 530/359; 536/23.5; 435/320.1; 514/7.4; 514/44 R; 514/4.6; 435/34; 435/7.1; 435/6.15; 530/388.4; 435/325; 530/387.9; 800/14; 800/15; 435/6.12; 424/529; 424/531

(57) ABSTRACT

An isolated human Apolipoprotein L-I corresponding to a wild type human Apolipoprotein sequence is modified by a deletion at its C-terminal end.

### FIELD OF THE INVENTION

The present invention is in the field of Molecular Biology and is related to Apolipoprotein L-I variants sequence(s) (c-terminal mutant of Apolipoprotein L-I (apoL1)) and its/their pharmaceutical (therapeutical or prophylactic) use, especially for a treatment and/or a prevention of diseases induced in mammals, especially in human, preferably infections induced by Trypanosoma, especially African Trypanosoma, more particularly Trypanosoma brucei rhodesiense and/or Trypanosoma brucei gambiense.

### BACKGROUND OF THE INVENTION AND STATE OF THE ART

Apolipoprotein L-I (apoL1) is a human-specific serum protein that kills Trypanosoma brucei through ionic pore formation in endosomal membranes of the parasite. The T. brucei subspecies rhodesiense and gambiense resist this lytic activity and can infect humans, causing sleeping sickness. In the case of T. b. rhodesiense resistance to lysis involves interaction of the Serum Resistance-Associated (SRA) protein with the C-terminal helix of apoL1.

Normal human serum (NHS) is able to kill T. b. brucei, but not T. b. rhodesiense and T. b. gambiense. The lytic factor was identified as being apoL1. This protein is associated with HDL particles that are efficiently taken up by the parasite through specific binding to a haptoglobin-hemoglobin surface receptor, due to the simultaneous presence of haptoglobin-related protein (Hpr) acting as a ligand in these particles. Trypanosome lysis results from anionic pore formation by apoL1 in the lysosomal membrane of the parasite. Resistance to lysis has only been studied in case of T. b. rhodesiense, where it was shown to depend on a parasite protein termed SRA. As synthesis of SRA only occurs after transcriptional activation of a given Variant Specific Glycoprotein (VSG) gene expression site from a repertoire of 10-20 candidates, T. b. rhodesiense clones can be either sensitive or resistant to NHS depending on which expression site is active. The mechanism by which SRA inhibits the activity of apoL1 is unclear. Direct coil-coiling interaction between the C-terminal α-helix of apoL1 and the N-terminal α-helix of SRA was demonstrated in vitro, but in vivo only evidence for tight co-localization between the two proteins was obtained. Total deletion of the C-terminal helix appeared to confer toxic activity to recombinant apoL1 on T. b. rhodesiense, suggesting that, in vivo, SRA neutralizes apoL1 through interaction with its C-terminal domain. However, the trypanolytic effect of this deleted apoL1 was weak and incomplete. Moreover, data obtained following transgenic expression of a similarly truncated apoL1 in mice suggested that its trypanolytic potential was lost in vivo.

### AIMS OF THE INVENTION

The present invention aims to propose a new pharmaceutical composition comprising one or more Apolipoprotein variant(s) (in the form of an amino acid sequence, or a nucleotide sequence(s), a vector, a cell, a blood sample and/or particles including HDL particles) or an inhibitor of this Apolipoprotein that could be administrated to mammals, especially to humans to cure and/or prevent Trypanosoma infections (especially T. b. rhodesiense) and related diseases (possibly in the treatment and/or prevention of glomerulosclerosis including focal segmental glomerulosclerosis (FSGS) cause of idiopathic nephrotic syndrome, HIV associated nephropathy and hypertension associated end-stage kidney disease (ESKD) in these mammals, especially in humans.

### SUMMARY OF THE INVENTION

The present invention is related to a (an isolated) human Apolipoprotein L-I sequence (variant) corresponding to this wild type human Apolipoprotein sequence (SEQ.ID.NO.1, SEQ.ID.NO.4 or SEQ.ID.NO.7) modified by (which comprises) a deletion of 1, 2, 3, 4, 5, 6, 7, 8, 9, 10, 11, 12, 13, 14, 15, 16, 17, 18, 19 or 20 of its last C-terminal amino acids.

More preferably, the Apolipoprotein L-I sequence (variant) according to the invention is the wild type human Apolipoprotein sequence, but exhibiting N388/Y389 deletion (a deletion of two amino acids located at its last C-terminal amino acids).

Alternatively, the Apolipoprotein L-I sequence (variant) according to the invention is the wild type human Apolipoprotein sequence, but exhibiting S342G/I384M mutations.

Preferably, the human Apolipoprotein (variant) according to the invention presents a sequence which is selected from the group consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8.

Another aspect of the present invention is related to an inhibitor, such as a (monoclonal) antibody or an specific hypervariable portion thereof, including nanobodies, specifically recognizing (and possibly neutralizing its function) a protein sequence of the invention, preferably selected from the group consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8 (and preferably not recognizing SEQ.ID.NO.1, SEQ.ID.NO.4 or SEQ.ID.NO.7), and the (hybridoma) cell producing this inhibitor, preferably this (monoclonal) antibody or its portion.

Another aspect of the present invention is related to a polynucleotide sequence encoding the Apolipoprotein L-I according to the invention and a vector comprising the (amino acid sequence of) Apolipoprotein L-I of the invention or its corresponding (coding) polynucleotide sequence.

Another aspect of the present invention is related to a cell transformed by this amino acid or polynucleotide sequence according to the invention and/or expressing the (recombinant and modified) Apolipoprotein L-I according to the invention; this cell is preferably a (non human embryonic) mammal cell, possibly grown in vitro.

Another aspect of the present invention is related to a diagnostic kit comprising means and media to identify whether a subject (including a human patient) comprises in his genome (and is expressing) the ApoL-I according to the invention (being preferably SEQ.ID.NO.2, SEQ.ID.NO.3, SEQ.ID.NO.5, SEQ.ID.NO.6, SEQ.ID.NO.8, or SEQ.ID.NO.9 or, more preferably being SEQ.ID.NO.2, SEQ.ID.NO.5 or SEQ.ID.NO.8.).

In this diagnostic kit, the preferred means are selected from the group consisting of nucleotide probes (nucleotide sequence) or antibodies (including specific hypervariable portions thereof or nanobodies) possibly present upon (fixed to a solid support to form) a micro-array or primers able to amplify corresponding sequences by genetic amplification means (PCR, LCR, etc) able to identify these Apolipoprotein L-I variants (of the invention), inhibitors or markers, such as antibodies or specific hypervariable portions thereof (including nanobodies), specifically recognizing these Apolipoprotein L-I variants (being preferably SEQ.ID.NO.2, SEQ.ID.NO.3, SEQ.ID.NO.5, SEQ.ID.NO.6, SEQ.ID.NO.8, or SEQ.ID.NO.9 or, more preferably being SEQ.ID.NO.2, SEQ.ID.NO.5 or SEQ.ID.NO.8. (and more preferably not recognizing Apolipoprotein L-I of SEQ.ID.NO.1, SEQ.ID.NO.4 and/or SEQ.ID.NO.7)) and Trypanosoma brucei rhodesiense culture (possibly in conjunction with the ApoL1 of the invention as positive control for lysis).

The preferred kit may further comprises recombinant SRA sequence fixed upon a solid support (possibly in conjunction with the ApoL1 of the invention as negative control for binding).

The present invention further discloses a diagnostic (method) comprising the step of:
extracting a (DNA or RNA) nucleotide sequence from a biological sample obtained from a patient;
identifying if this DNA or RNA sequence is a variant in Apolipoprotein L-I, preferably being a DNA or a RNA variant nucleotide sequence encoding a protein sequence being selected from the group consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8;
deducing whether this patient is carrying the variance in Apolipoprotein L-I and possibly whether this patient is homozygote or heterozygote for ApoL1 variation.

Alternatively, a related diagnostic method comprises the step of:
analysing a blood sample for variant in Apolipoprotein L-I (being preferably a protein sequence being selected from the group consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8) through binding to the specific antibodies, hyper variable portions thereof or nanobodies of the present invention;
deducing whether this patient is carrying the variance in Apolipoprotein L-I and possibly whether this patient is homozygote or heterozygote for ApoL1 variation.

Alternatively, a related diagnostic method comprises the step of:
analysing a blood sample for variant in Apolipoprotein L-I through its capacity to lysate a Trypanosoma brucei rhodesiense culture;
deducing whether this patient is carrying the variance in Apolipoprotein L-I.

Alternatively (but less preferably), a related diagnostic method comprises the step of:
analysing a blood sample for variant in Apolipoprotein L-I through its absence of binding to SRA;
deducing whether the patient is carrying the variance in Apolipoprotein L-I. This method can be combined with the others above-described preferred diagnostic methods of the present invention.

Another aspect of the present invention is related to a pharmaceutical composition (including a vaccine) comprising an adequate pharmaceutical carrier (or diluent and possibly one or more adequate adjuvant(s)) and a sufficient amount of an element selected from the group consisting of the Apolipoprotein L-I (amino acid sequence) according to the invention, the inhibitor (preferably the antibody or its portion) according to the invention, the polynucleotide according to the invention, the vector according to the invention or the cell (possibly in the form of a pharmaceutically-acceptable lysate or lyophilisate) according to the invention; preferably, this pharmaceutical composition (vaccine) is used in (for) a treatment and/or a prevention of diseases induced in mammals (by Trypanosoma brucei; more preferably by Trypanosoma brucei rhodesiense)), being preferably humans; and wherein the Apolipoprotein L-I of the invention is preferably capable impeding its interaction with the Serum Associated protein (SRA) and/or to act despite having interacted with SRA.

Another aspect of the invention is a composition comprising from 100 pg/ml to 10 μg/ml of the Apolipoprotein L-I of the invention (consisting preferably of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8).

A related aspect of the invention is a blood sample (preferably a serum) or extract thereof comprising the Apolipoprotein L-I of the invention (consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8).

Preferably this blood sample or extract thereof is in the form of (HDL) particles.

Advantageously, this blood sample (serum or extract preferably in the form of (HDL) particles) is for use as a medicament.

Preferably, this blood sample (serum or extract, including in the form of (HDL) particles) is for use in the treatment or prevention of Trypanosoma infections.

Preferably this blood sample (serum or extract preferably in the form of (HDL) particles) is for use in (or for the manufacture of a medicament for) the treatment of Trypanosoma brucei infections.

More preferably, this blood sample (serum or extract, preferably in the form of (HDL) particles) is for use in (or for the manufacture of a medicament for) the treatment or prevention of Trypanosoma brucei rhodesiense infections.

Possibly, the Apolipoprotein L-I of the invention is obtained (and/or purified) from blood samples comprising it.

Alternatively, the Apolipoprotein L-I of the invention is obtained after in vitro fermentation using the transfected cells of the invention.

Another aspect of the present invention is related to a non-human genetically modified mammal, which is expressing the Apolipoprotein L-I according to the invention (being preferably selected from the group consisting of SEQ.ID.NO.2, SEQ.ID.NO.3, SEQ.ID.NO.5, SEQ.ID.NO.6, SEQ.ID.NO.8, and SEQ.ID.NO.9 and more preferably being from the group consisting of SEQ.ID.NO.2, SEQ.ID.NO.5 and SEQ.ID.NO.8) or which may comprise the polynucleotide, the vector or the cell according to the invention or which may express the synthesis of the amino acid sequence of the Apolipoprotein L-I of the invention.

Preferably, this non-human genetically modified mammal is a genetically modified cattle, preferably genetically modified cow, which could be resistant or tolerant to infection(s) induced by Trypanosoma and non or slowly affected by the related diseases (NAGANA), preferably infection(s) and disease(s) induced by Trypanosoma brucei brucei, Trypanosoma brucei rhodesiense, trypanosoma congolense, trypanosoma evansi and/or trypanosoma vivax.

Alternatively, this non-human genetically modified mammal is a genetically modified rodent possibly used in research as a model for a disease (such as glomerulosclerosis), like a mouse or a rat.

A last aspect of the invention is related to the treatment or prevention of glomerulosclerosis, especially focal segmental glomerulosclerosis (FSGS).

Possibly, the present invention provides for the use of a (specific) inhibitor of the function of a the ApoL1 of the invention (preferably selected from the group consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8) as a medicament.

Preferably, the present invention provides for the use of a (specific) inhibitor (preferably an (monoclonal) antibody, a specific hypervariable portion thereof or a nanobody) of the function of a protein sequence selected from the group consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8 for the treatment and/or the prevention of glomerulosclerosis.

Advantageously, the present invention provides for the use of antibodies (including specific hypervariable portions thereof or nanobodies) specifically recognizing (and preferably neutralizing its function) a protein sequence selected from the group consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8 for use as a medicament.

Preferably, the specific inhibitor (more preferably a neutralizing (monoclonal) antibody (including specific hypervariable portions thereof or nanobodies) is for use in the treatment of glomerulosclerosis, including focal segmental glomerulosclerosis (FSGS), in patients expressing the Apolipoprotein L-I of the present invention (consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8) and more preferably not expressing SEQ.ID.NO.1, SEQ.ID.NO.4 or SEQ.ID.NO.7.

Advantageously, the present invention provides for drugs to reduce blood pressure (antihypertensive) and/or blood cholesterol content for use in (or for the manufacture of a medicament for) a treatment and/or for a prevention of glomerulosclerosis (including Focal segmental glomerulosclerosis (FSGS)) cause of idiopathic nephrotic syndrome, HIV associated Nephropathy and hypertension-associated end-stage kidney disease (ESKD) mostly observed in African Americans) for patients expressing the Apolipoprotein L-I of the present invention (preferably consisting of SEQ.ID.NO.2, SEQ.ID.NO.3, SEQ.ID.NO.5, SEQ.ID.NO.6, SEQ.ID.NO.8, and SEQ.ID.NO.9, being more preferably selected from the group consisting of SEQ.ID.NO.2, SEQ.ID.NO.5 and SEQ.ID.NO.8) and more preferably not expressing SEQ.ID.NO.1, SEQ.ID.NO.4 or SEQ.ID.NO.7.

Preferably, the drugs to reduce blood pressure (antihypertensive) according to the invention are selected from the group consisting of: angiotensin-converting inhibitors (such as captopril, enalapril, fosinopril (Monopril®), lisinopril (Zestril®), quinapril and ramipril (Altace®)), angiotensin II receptor antagonists (such as telmisartan (Micardis, Pritor), irbesartan (Avapro), losartan (Cozaar®), valsartan (Diovan®), candesartan (Amias®), olmesartan (Benicar®, Olmetec®), calcium channel blockers (such as nifedipine (Adalat®) amlodipine (Norvasc®), diltiazem, verapamil, diuretics (such as bendroflumethiazide, chlorthalidone, hydrochlorothiazide) or a mixture thereof.

Preferably, the drugs to reduce blood cholesterol levels according to the invention are selected from the group consisting of statins (most prominently rosuvastatin, atorvastatin, simvastatin, or pravastatin), cholesterol absorption inhibitors (ezetimibe), fibrates (gemfibrozil, bezafibrate, fenofibrate or ciprofibrate), vitamin B3 (niacin), bile acid sequestrants (colestipol, cholestyramine) or a mixture thereof.

Alternatively, blood cholesterol levels can be reduced by appropriate diet, such as cholesterol-reduced feed and/or fat (especially saturated and/or trans)-reduced feed.

The present invention will be described in more details in the following detailed description of the invention in reference to the enclosed figures presented as non limited illustrations of the present invention.

### SHORT DESCRIPTION OF THE FIGURES

FIG. 1: Trypanolytic potential of apoL1 variants on NHS-resistant (ETat 1.2R; SRA+: upper panel) and NHS-sensitive (ETat 1.2S; SRA−: lower panel) T. brucei ETat 1.2 clones and titration of trypanolytic activity in plasma samples after overnight incubation (100%=control incubation in fetal calf serum without plasma; hom, het=homozygous and heterozygous mutations, respectively; G1 stands for S342G/I384M mutation, while G2 stands for N388/Y389 deletion).

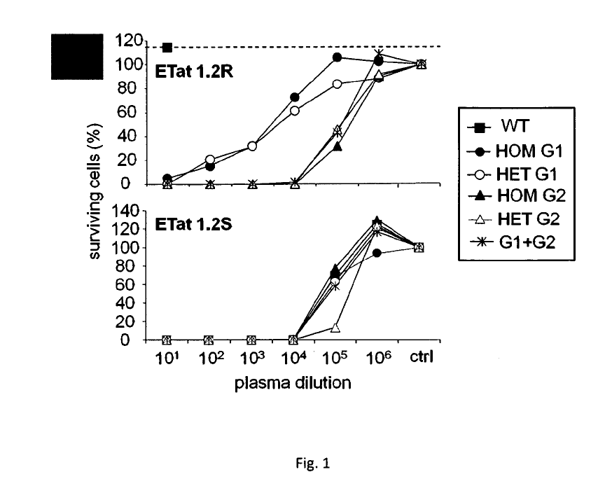

FIG. 2: ApoL1 content of various plasma samples before and after affinity chromatography through SRA column (NHS=normal human serum; WT=wild type apoL1 ; S=serine 342; G=glycine 342; I=isoleucine 384; M=methionin384; i=insertion of N388/Y389; d=deletion of N388/Y389).

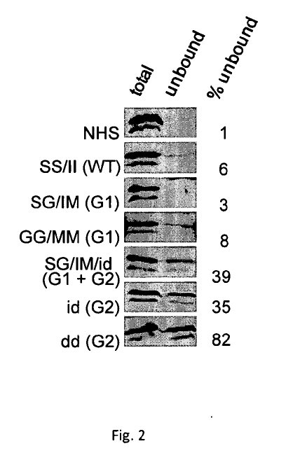

FIG. 3: Trypanolytic activity of several recombinant apoL1 variants after overnight incubation (FCS=fetal calf serum) on resistant (R) and sensitive (S) clones of Trypanosoma brucei.

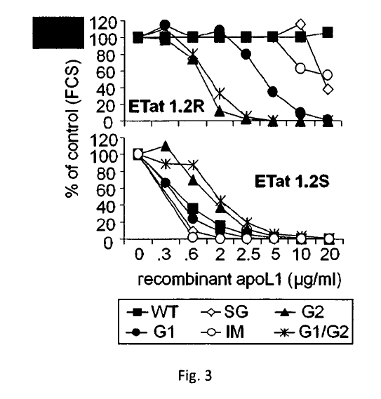

FIG. 4:. Kinetics of trypanolysis of resistant T. brucei rhodesiense by 20 μg/ml recombinant apoL1 variants, in the presence or absence of 25 μM chloroquine (clq).

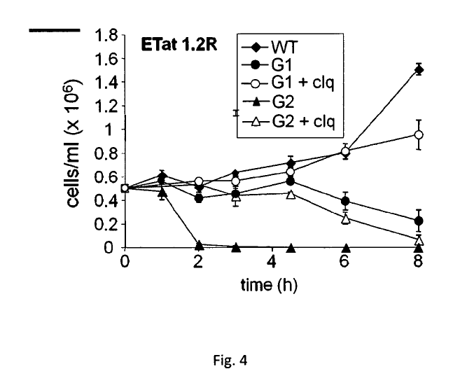

FIG. 5: Phenotype of ETat1.2R trypanosomes (T. brucei rhodesiense) incubated with various recombinant apoL1 (20 pg/ml; 1h30 and 6h incubation, for G1 and G2 respectively; the arrows point to the swelling lysosome).

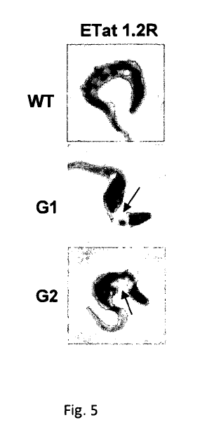

### DETAILED DESCRIPTION OF THE INVENTION

The serum protein apolipoprotein L-I (apoL1 ) is responsible for human innate immunity against Trypanosoma brucei brucei, because this protein kills the parasite by generating ionic pores in the lysosomal membrane. Two T. brucei subspecies (T. b. rhodesiense and T. b. gambiense) can resist apoL1 and therefore, infect humans and cause sleeping sickness. In T. b. rhodesiense resistance to human serum is linked to interaction of the Serum Resistance-Associated protein with the C-terminal region of apoL1. Mutations targeted to this region reduced its interaction with SRA, while preserving the activity of the ionic pore-forming domain. The inventors identified variants that did not bind to SRA, but acquired the ability to efficiently kill T. b. rhodesiense.

However, the inventors previously showed that mutants they produced in the L370-L392 leucine zipper lost in vitro trypanolytic activity. Mutants in the conserved G361-5364 motif still interacted with SRA, but lost trypanolytic potential in some cases.

The inventors analyzed the effects of various naturally-occurring (as well as artificial ones) deletions and mutations in the C-terminal domain of apoL1 on the trypanolytic potential of this protein against T. b. brucei and T. b. rhodesiense.

The inventors further treated patients suffering from Trypanosoma infection (Trypanosoma b. rhodesiense) with blood samples (serum or HDL fractions) obtained from patients expressing ApoL1 variants (being homozygotes or heterozygotes).

The inventors observed that Trypanosoma were killed in vivo, even when using elevated dilutions of these blood samples, resulting into the prevention of sleeping sickness in patients infected with b. rhodesiense. The inventors further noticed no renal toxicity, despite the injection of this variant of ApoL1 protein.

The inventors then screened from patients that carry variants of ApoL1 (patients that express the ApoL1 of the invention, being heterozygotes or, more preferably, homozygotes) and treat them in order to prevent (treat) the renal symptom associated with these variant.

In addition, rodent expressing the ApoL1 of the present invention were investigated for their renal pathologies and for the development of corresponding treatments.

### Material and Method

Unless stated otherwise, the experiments, including Trypanaosoma culture and the tests of human sera for their lytic activities, were carried-out in a manner similar to the ones already published: Locordier L. et al., 2009; C-terminal mutants of apolipoprotein L-I efficiently kill both Trypanosoma brucei brucei and Trypanosoma brucei rhodesiense; PLoS Pathog. 2009 December; 5(12):e1000685.

The sera obtained from patients with variant ApoL1 were diluted from 1000 to 100000 times and showed lytic activity even at these high dilutions.

More precisely, the inventors tested the variants of ApoL1 at concentrations ranging from 80 pg/ml to 20 μg/ml and observed in every case a lytic activity for every sera comprising SEQ.ID.NO.2 and for the majority of sera comprising SEQ.ID.NO.3.

The inventors used preferably the ApoL1 of the invention at about 10 ng/ml to about 20 μg/ml and still more preferably at about 2 μg/ml to about 10 μg/ml.

1.-24. (canceled)

25. An isolated human Apolipoprotein L-I corresponding to the wild-type human Apolipoprotein sequence (SEQ.ID.NO.1, SEQ.ID.NO.4 or SEQ.ID.NO.7) modified by a deletion of 1, 2, 3, 4, 5, 6, 7, 8, 9, 10, 11, 12, 13, 14, 15, 16, 17, 18, 19 or 20 of its last C-terminal amino acids.

26. An isolated human Apolipoprotein L-I, which is selected from the group consisting of SEQ.ID.NO.3, SEQ.ID.NO.6, and SEQ.ID.NO.9.

27. The Apolipoprotein L-I according to claim 25, which is selected from the group consisting of SEQ.ID.NO.2, SEQ.ID.NO.5 and SEQ.ID.NO.8.

28. A blood sample or blood extract comprising the Apolipoprotein L-I according to claim 25.

29. The blood sample of claim 28 being a serum.

30. The blood sample extract of claim 28 being an HDL particle.

31. A polynucleotide encoding the Apolipoprotein L-I according to claim 25.

32. A vector comprising Apolipoprotein L-I according to claim 25 or the polynucleotide encoding the Apolipoprotein L-I according to claim 25.

33. A cell transformed by a vector comprising Apolipoprotein L-I according to claim 25 or the polynucleotide encoding the Apolipoprotein L-I according to claim 25 and/or expressing the Apolipoprotein L-I.

34. A pharmaceutical composition comprising an adequate pharmaceutical carrier or diluent and a sufficient amount of the Apolipoprotein L-I according to claim 25, a blood sample or a blood sample extract comprising the Apolipoprotein L-I, the polynucleotide encoding the Apolipoprotein L-I, a vector comprising the Apolipoprotein L-I or the polynucleotide encoding the Apolipoprotein L-I, or the cell comprising Apolipoprotein L-I or the polynucleotide encoding the Apolipoprotein L-I and/or expressing the Apolipoprotein L-I.

35. The pharmaceutical composition according to the claim 34 for use in the treatment and/or the prevention of diseases induced in human by Trypanosoma brucei.

36. The pharmaceutical composition according to the claim 34 for use in the treatment and/or the prevention of diseases induced in human by Trypanosoma brucei rhodesiense.

37. A method of treatment and/or prevention of a disease related to infection by trypanosoma affecting a mammal, which comprises the step of administrating a sufficient amount of the pharmaceutical composition of claim 34 to the mammal to reduce and/or suppress the symptoms of the disease in the mammal.

38. The Method of claim 37, wherein the mammal is a human.

39. A diagnostic kit comprising:
nucleotide probes able to identify Apolipoprotein L-I variants; or antibodies specifically recognizing Apolipoprotein L-I variants according to claim 25, and/or
Trypanosoma brucei rhodesiense culture
and
means to identify whether a subject is expressing the Apolipoprotein L-I.

40. The kit of claim 39 further comprising recombinant SRA bound on a solid support.

41. A kit comprising
nucleotide probes able to identify Apolipoprotein L-I variants; or
antibodies specifically recognizing Apolipoprotein L-I variants according to claim 25, and/or
Trypanosoma brucei rhodesiense culture
and
means to identify whether a subject is expressing the Apolipoprotein L-I, the Apolipoprotein L-I.

42. An Antibody (preferably a monoclonal antibody) or a specific hypervariable portion thereof specifically recognizing Apolipoprotein L-I according to claim 25 and not recognizing Apolipoprotein L-I of SEQ.ID.NO.1, SEQ.ID.NO.4 and/or SEQ.ID.NO.7.

43. An inhibitor directed to the protein according to claim 25, for use in the treatment and/or the prevention of glomerulosclerosis.

44. A Blood-lowering cholesterol and/or antihypertensive for use in the treatment and/or for the prevention of glomerulosclerosis in patients expressing the Apolipoprotein L-I according to claim 25.

45. A pharmaceutical compound selected from the group consisting of angiotensin-converting inhibitors, angiotensin II receptor antagonists, calcium channel blockers, diuretics, statins, cholesterol absorption inhibitors, vitamin B3 and bile acid sequestrants for use in the treatment and/or for the prevention of glomerulosclerosis in patients expressing the Apolipoprotein L-I according to claim 25.

46. A non-human genetically modified mammal, which is expressing the Apolipoprotein L-I according to claim 25.

47. The non-human genetically modified mammal of claim 46, wherein the mammal is a rodent.

48. The non-human genetically modified mammal of claim 46, wherein the mammal is a cattle.
